(19) United States
(12) Patent Application Publication
(10) Pub. No.: US 2025/0009899 A1
(43) Pub. Date: Jan. 9, 2025

(54) FUSION PROTEINS CONTAINING IL21 MUTEINS FOR TREATING CANCER AND VIRAL DISEASES

(71) Applicant: Bluejay Therapeutics, Inc., San Mateo, CA (US)

(72) Inventors: Hassan Javanbakht, San Francisco, CA (US); Hilario J. Ramos, San Mateo, CA (US); Yue Liu, HAYWARD, CA (US); Matthew Hoag, HAYWARD, CA (US); Bo Wang, HAYWARD, CA (US)

(21) Appl. No.: 18/762,555

(22) Filed: Jul. 2, 2024

Related U.S. Application Data
(60) Provisional application No. 63/572,826, filed on Apr. 1, 2024.
(60) Provisional application No. 63/511,810, filed on Jul. 3, 2023.

(51) Int. Cl. A61K 47/68; A61P 35/00; C07K 16/28

(52) U.S. Cl. CPC A61K 47/6813; A61K 47/6849; A61P 35/00; C07K 16/2818

(57) ABSTRACT

A fusion protein includes an anti-PD1 antibody portion having a C-terminus, and an IL21 mutein conjugated with the anti-PD1 antibody portion with its C-terminus, wherein the IL21 mutein contains at least one of the two mutations relative to the wild-type IL21: (1) one of K72Y substitution, K72M substitution, and K72Q substitution, and (2) 75-80 deletion. In some examples, the anti-PD1 antibody can adopt a ETYY format, and can include a first heavy chain comprising a VH comprising a sequence of SEQ ID NO:11, and a second heavy chain comprising a VH comprising a sequence selected from the sequences of SEQ ID NOS:8-10. Pharmaceutical compositions including the fusion protein, nucleic acid coding the fusion protein, vectors including the nucleic acid, and methods of treating diseases using the fusion protein are also provided.

### INCORPORATION OF SEQUENCE LISTING

This application includes a Sequence Listing which has been submitted in XML format via Patent Center, named “Bluej005.XML” which is 28 KB in size and created on Jun. 28, 2024. The contents of the Sequence Listing are incorporated herein by reference in their entirety.

### BACKGROUND

Interleukin-21 (IL-21, or IL21) is a pleiotropic cytokine belonging to the common gamma chain (γc) family, with critical roles in the regulation of both innate and adaptive immune responses. Discovered in 2000, IL-21 is mainly produced by activated CD4+ T cells, particularly T follicular helper (Tfh) cells and natural killer T (NKT) cells, and acts on various immune cell types, including T cells, B cells, NK cells, and dendritic cells. This cytokine has been implicated in modulating numerous immunological processes, such as T cell differentiation, B cell proliferation, and antibody production, as well as enhancing cytotoxic activity of NK cells. The potential therapeutic applications of IL-21 have attracted significant attention in recent years, with a focus on its capacity to modulate immune responses in the context of chronic viral infections and cancer.

Chronic hepatitis B virus (HBV) infection remains a major public health issue, affecting millions of individuals worldwide and causing significant morbidity and mortality due to liver cirrhosis and hepatocellular carcinoma. Current antiviral therapies, including nucleos(t)ide analogs and interferon-alpha, have limited efficacy in achieving a functional cure for the majority of patients. IL-21 has emerged as a promising candidate for the treatment of chronic HBV infection, given its ability to boost antiviral immune responses and promote viral clearance. Studies have demonstrated that IL-21 can enhance the function of HBV-specific T cells, augmenting their proliferation, cytotoxic activity, and cytokine production. Moreover, IL-21 has been shown to suppress HBV replication in human hepatocytes and animal models, highlighting its potential as a novel therapeutic agent for achieving a functional cure in chronic HBV patients.

IL-21 has also shown promise in the field of oncology, given its capacity to stimulate potent antitumor immune responses. Preclinical studies have demonstrated that IL-21 can enhance the cytotoxic activity of NK cells and CD8+ T cells, leading to the eradication of tumor cells in various cancer models. In addition, IL-21 can promote the differentiation of T cells towards a Th1 phenotype, which is associated with improved antitumor immunity. Clinical trials investigating the safety and efficacy of IL-21 as a monotherapy or in combination with other immunotherapies have shown encouraging results in patients with metastatic melanoma and renal cell carcinoma. Further research is needed to fully elucidate the therapeutic potential of IL-21 in cancer and to optimize its application in combination with other emerging immunotherapeutic approaches.

IL-21 has also emerged as a potential therapeutic agent for human immunodeficiency virus (HIV) infection, due to its immunomodulatory properties and ability to enhance the function of virus-specific immune responses. HIV infection is characterized by progressive CD4+ T cell depletion, immune dysregulation, and the establishment of a viral reservoir, which presents a major barrier to achieving a functional cure. Research has demonstrated that IL-21 can promote the survival, proliferation, and function of CD4+ and CD8+ T cells in HIV-infected individuals, as well as boost the cytotoxic activity of NK cells, thereby enhancing antiviral immunity. Additionally, IL-21 has been shown to exert a direct antiviral effect on HIV replication in vitro, and its administration in simian immunodeficiency virus (SIV)-infected non-human primates resulted in reduced viral loads and improved immune reconstitution. These findings suggest that IL-21 may hold promise as a novel immunotherapeutic approach for the treatment of HIV, either alone or in combination with antiretroviral therapy and other immune-based interventions.

### SUMMARY

In one aspect of the disclosed subject matter, a fusion protein is provided, which comprises an anti-PD1 antibody portion having a C-terminus, and an IL21 mutein conjugated with the anti-PD1 antibody portion with its C-terminus, wherein the IL21 mutein contains one or both of the following mutations relative to the wild-type IL21: (1) one of K72Y substitution, K72M substitution, and K72Q substitution, and (2) 75-80 deletion. For example, the IL21 mutein can comprise an amino acid sequence selected from the group consisting of SEQ ID NOS:2-6.

In some embodiments of the fusion protein, the IL21 mutein contains both of (1) one of K72Y substitution, K72M substitution, and K72Q substitution, and (2) 75-80 deletion. In some embodiments, the IL21 mutein only contains one of the two mutations relative to the wild-type IL21: (1) one of K72Y substitution, K72M substitution, and K72Q substitution, and (2) 75-80 deletion.

In some embodiments, the anti-PD1 antibody has an IgG1 format and comprises a first heavy chain and second heavy chain, the first heavy chain comprising a T366Y mutation and the second heavy chain comprising a Y407T mutation.

In some embodiments, the anti-PD1 antibody has an IgG1 format and comprises a first heavy chain and second heavy chain, the first heavy chain containing K360, S354Y and T366Y mutations, and the second heavy chain containing G347E, Y349 and Y407T mutations.

In some embodiments, the anti-PD1 antibody comprises a first heavy chain comprising a VH comprising a sequence of SEQ ID NO:7. In some of these embodiments, the anti-PD1 antibody further comprises a second heavy chain comprising a VH comprising a sequence selected from the sequences of SEQ ID NOS:8-10.

In some embodiments, the anti-PD1 antibody comprises a first heavy chain comprising a VH comprising a sequence of SEQ ID NO:11. In some of these embodiments, the anti-PD1 antibody further comprises a second heavy chain comprising a VH comprising a sequence selected from the sequences of SEQ ID NOS 8-10.

In some embodiments, the fusion protein comprises a first heavy chain including a mutant Fc comprising a sequence of SEQ ID NO:13, and a second heavy chain including a mutant Fc comprising a sequence of SEQ ID NO:12.

In some embodiments, the Fc of the first heavy chain in the KIH structure comprises the sequence set forth in SEQ ID NO:15, and Fc of the second heavy chain in the KIH structure comprises the sequence set forth in SEQ ID NO:14.

In some embodiments, the Fc of the first heavy chain in the ETYY structure comprises the sequence set forth in SEQ ID NO:17, and Fc of the second heavy chain in the ETYY structure comprises the sequence set forth in SEQ ID NO:16.

In some embodiments, only the first heavy chain is conjugated with the IL21 mutein and the second heavy chain is not conjugated with any IL21 mutein.

In some embodiments of the fusion protein (or antibody cytokine fusion), the anti-PD1 antibody portion and the IL-21 mutein are conjugated via a linker therebetween. The linker can be connected to the C-terminus of an anti-PD1 antibody heavy chain, and then connected to the IL-21 mutein, as illustrated in FIGS. 3e and 3f. In some embodiments, the linker comprises (GₙS)ₓ, wherein n is an integer between 2 and 4 inclusive, and x is an integer between 1 and 4 inclusive. In some embodiments, the anti-PD1 antibody portion (e.g., a heavy chain of the anti-PD1 antibody) is directly connected to the IL21 mutein without a linker, as illustrated in FIGS. 1a-1f, 2a-2f, and 3a-3d.

In some embodiments of the fusion protein, the first heavy chain comprises a sequence selected from the groups consisting of SEQ ID NOS:17-25, e.g., SEQ ID NOS, 20, 22, 23, 24, 25. The IL21 mutein conjugated with the first heavy chain can comprise a sequence selected from the group consisting of SEQ ID NOS:2-6, e.g., SEQ ID NO:6.

In some embodiments, the first heavy chain comprises a Fc region comprising the sequence selected from the groups consisting of SEQ ID NOS:17-22. In some of these embodiments, the first heavy chain can comprise a VH having the sequence of SEQ ID NO:11, and the second heavy chain can comprise a VH having the sequence of one of SEQ ID NOS:8-10, e.g., SEQ ID NO:10.

In a further aspect, the present disclosure provides a nucleic acid encoding the fusion protein disclosed herein.

In a further aspect, the present disclosure provides a vector comprising the nucleic acid.

In a further aspect, the present disclosure provides a pharmaceutical composition comprising a fusion protein disclosed herein and a pharmaceutically acceptable carrier.

In a further aspect, the present disclosure provides a method of treating a cancer of a subject, which comprises administering to the subject an effective amount of a fusion protein or of the pharmaceutical composition disclosed herein. The cancer can be a solid tumor, a hematological malignancy, or a lymphoid malignancy. For example, the cancer can include lung cancers, head and neck cancers, kidney cancers, breast cancers, brain cancers, melanoma and other skin cancers, ovarian cancers, liver cancers, pancreatic cancers, colon cancers, colorectal cancers, prostate cancers, gastrointestinal cancers, bladder cancers, blood cancers, lymphomas testicular cancers, gynecological cancers, and sarcomas.

In a further aspect, the present disclosure provides a method of treating a chronic viral infection of a subject, comprising administering to the subject an effective amount of a fusion protein or of the pharmaceutical composition described herein. In some embodiments, the chronic viral infection can be HBV infection or HIV infection. In other embodiments, the chronic viral infection includes Herpes Simplex Virus Type 1 (HSV-1), Herpes Simplex Virus Type 1 (HSV-2), Kaposi's Sarcoma-Associated Herpesvirus (KSHV), Human Herpesvirus 7 (HHV-7), Human Herpesvirus 6 (HHV-6), Cytomegalovirus (CMV), Epstein-Barr Virus (EBV), Varicella-Zoster Virus (VZV), Hepatitis D virus (HDV) and human papilloma virus (HPV)

### BRIEF DESCRIPTION OF THE DRAWINGS

FIGS. 1a-1f show example schematic structures of certain anti PD-1 x IL-21 mutein fusion proteins according to embodiments of the present invention.

FIGS. 2a-2f show further example schematic structures of certain anti PD-1 x IL-21 mutein fusion proteins according to embodiments of the present invention.

FIGS. 3a-3f show another set of example schematic structures of certain anti PD-1 x IL-21 mutein fusion proteins according to embodiments of the present invention, based on variations of Fc region of anti-PD-1 antibody structure shown in FIG. 2e.

FIG. 4a is a plot showing kinetics of tumor control by isotype control compared against an anti-PD-1/IL-21 mutein of the present invention in a humanized mouse model of colorectal cancer.

FIG. 4b is a plot showing percent Tumor Growth Inhibition (TGI) on days 11 and 20 post treatment with isotype compared against an anti-PD-1/IL-21 mutein of the present invention in a humanized mouse model of colorectal cancer.

### DETAILED DESCRIPTION

In one aspect of the present disclosure, a fusion protein (or protein fusion) is provided which comprises an anti-PD1 antibody portion having a C-terminus, and an IL21 mutein conjugated with the anti-PD1 antibody portion with its C-terminus. They are generally referred to as anti PD-1 x IL-21 (or anti PD-1/IL-21, or anti PD-1-IL-21) fusion proteins herein. Wild type human IL-21 amino acid sequence is provided herein as SEQ ID NO:1.

Relative to the wild-type human IL21, the IL21 muteins herein are designed to have varying reduced affinities to IL-21 receptor (IL-21R). The fusion proteins herein are unresponsive to IL-21R binding unless PD-1 receptor co-expression is present. PD-1 is expressed as a function of immune cell activation and therefore is only present on immune cells within the diseased tissue or active site of inflammation. Thus, the anti PD-1/IL-21 mutein fusion proteins provided herein can provide selective IL-21 activation and improved safety and therapeutic index in patients with chronic viral infection or cancers. The selective activity can allow for increased plasma exposure and improved PD-1 blockade and IL-21R activation on immune subsets where disease is present. Therefore, the anti PD-1/IL-21 mutein fusion proteins described here can selectively deliver an immune activation to the site of disease while limiting engagement of non-PD-1 positive immune cells within peripheral blood, immune organs, or non-diseased tissue.

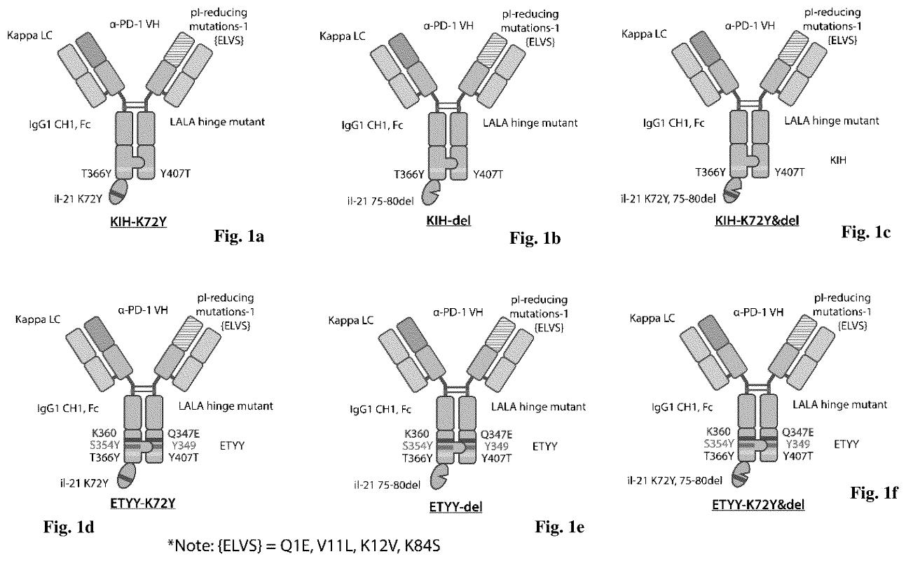

Various examples of fusion proteins having dual chain structures of the disclosed subject matter are shown in FIGS. 1-3. It is noted that the features of the fusion protein may be described by way of the structure or features their constituents or components, e.g., the antibody portion (including its sub-components, e.g., heavy chain, light chain, VH, Fc, CH3, etc.), the linker, and the IL21 mutein. The variations in the structure of different components of a fusion protein can be combined.

As shown in FIGS. 1-3, example structures of the fusion protein according to some embodiments of the present invention, the anti-PD1 antibody portion (also referred to simply as the anti-PD1 antibody) of the fusion protein can have an IgG1 format and comprises a first heavy chain and second heavy chain (and paired light chains, e.g., kappa light chains, respectively). While the fusion proteins depicted in FIGS. 1-3 include a conjugated IL21 mutein only on the first heavy chain of its two heavy chains, it is contemplated that the fusion protein of the disclosed subject matter could include a conjugated IL21 mutein in each of its two heavy chains.

In some embodiments of the fusion protein, the IL21 mutein in the fusion protein differs from the wild-type IL21 in that it contains at least one of the following mutations relative to the wild-type IL21: (1) K72Y, or K72M, or K72Q substitution, and (2) 75-80 deletion. For example, the IL21 mutein can have a single mutation of K72Y, K72M, or K72Q substitution, but not the 75-80 deletion. For another example, the IL 21 mutein can have the mutation of 75-80 deletion but not the K72 substitution. For yet another example, the IL21 mutein includes both a K72 substitution (K72Y, K72M or K72Q) and the 75-80 deletion.

The amino acid sequence of IL-21 mutant denoted as K72Y is provided in SEQ ID NO:2.

The amino acid sequence of IL-21 mutant denoted as 75-80 deletion is provided in SEQ ID NO:3.

The amino acid sequence of IL-21 mutant denoted as K72Y, 75-80 deletion is provided in SEQ ID NO:4.

The amino acid sequence of IL-21 mutant denoted as K72M, 75-80 deletion is provided in SEQ ID NO:5.

The amino acid sequence of IL-21 mutant denoted as K72Q, 75-80 deletion is provided in SEQ ID NO:6.

In some embodiments, the two heavy chains of the anti-PD1 portion of the fusion protein can be in a “knob-into-hole” (or KIH) structure. For example, the first heavy chain of the anti-PD1 portion can comprise a T366Y mutation, and the second heavy chain comprises a Y407T mutation as shown in FIG. 1 as KIH-K72Y, KIH-del, and KIH-K72Y&del.

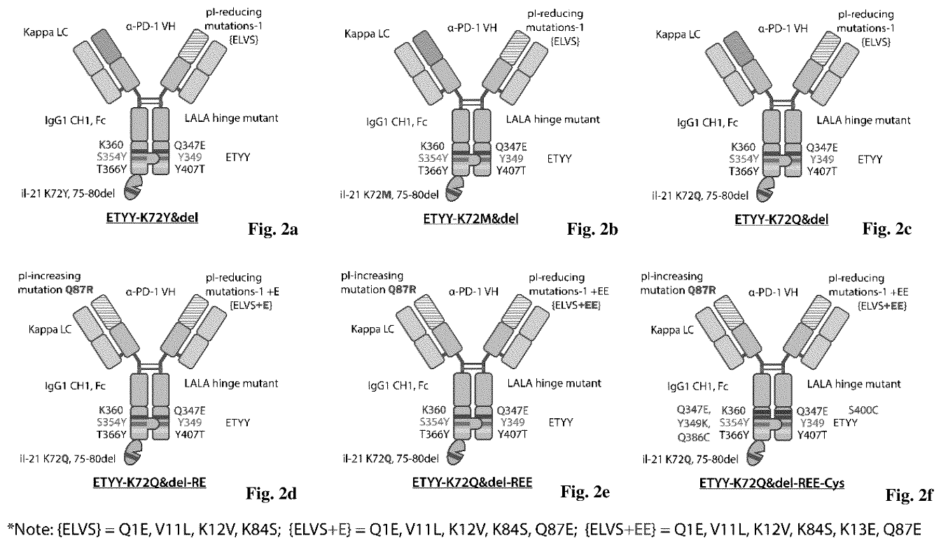

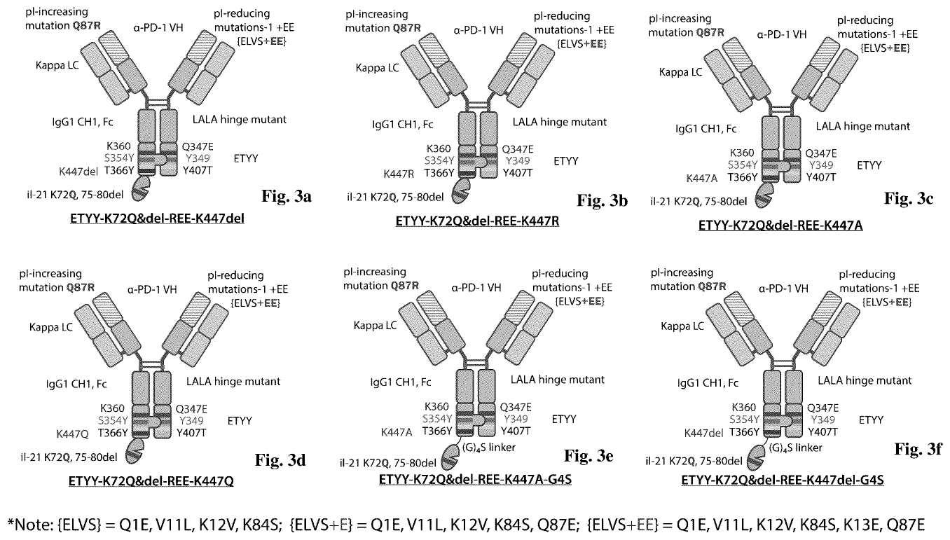

In some embodiments, the first heavy chain contains K360, S354Y and T366Y mutations, and the second heavy chain contains G347E, Y349 and Y407T mutations (also referred to as “ETYY” structure), as illustrated in FIG. 1 as ETYY-K72Y, ETYY-del, ETYY-K72Y&del, as well as structures illustrated in FIGS. 2 and 3.

In some embodiments, the Fc (CH3) of the first heavy chain in the KIH structure comprises the sequence set forth in SEQ ID NO:15, and Fc (CH3) of the second heavy chain in the KIH structure comprises the sequence set forth in SEQ ID NO:14.

In some embodiments, the Fc (CH3) of the first heavy chain in the ETYY structure comprises the sequence set forth in SEQ ID NO:17, and Fc (CH3) of the second heavy chain in the ETYY structure comprises the sequence set forth in SEQ ID NO:16.

In some embodiments, the anti-PD1 portion of the fusion protein comprises a hinge portion having a LALA substitutions, as illustrated in FIGS. 1-3.

In some embodiments of the fusion protein, the anti-PD1 antibody portion comprises a first heavy chain on the “knob” side of the dual chain comprising a VH having a sequence of the VH of pembrolizumab (or Keytruda) (wt Parental), i.e., SEQ ID NO:7. In some of these embodiments, the anti-PD1 antibody portion further comprises a second heavy chain on the “hole” side comprising a VH having the sequence of a mutant Keytruda VH protein in order to enlarge the pI difference between the two half antibody of an asymmetric bispecific IgG to enable better heterodimer formation and CMC production, as exemplified in FIGS. 1-3. In an example, such a mutant Keytruda VH has mutations Q1E, V11L, K12V, K84S (referred to as ELVS mutant) having a sequence of SEQ ID NO:8. In another example, such a mutant Keytruda VH has ELVS+E mutations (Q1E, V11L, K12V, K84S, Q87E) having a sequence of SEQ ID NO:9. In a further example, such a mutant Keytruda VH has ELVS +EE (Q1E, V11L, K12V, K84S, K13E, Q87E) mutations having a sequence of SEQ ID NO:10.

In some embodiments, the first heavy chain can include a mutant Keytruda VH, e.g., Q87R, whose sequence is set forth in SEQ ID NO:11, while the second chain includes a mutant Keytruda VH, such as ELVS+E, ELVS+EE, etc., as shown in FIGS. 2a-2f. In some embodiments, both of the first heavy chain and the second heavy chain include unmutated Keytruda VH.

In some embodiments, the second heavy chain can also include a mutant Fc (hIgG1-Fc containing S400C substitution), the CH3 sequence of which is set forth in SEQ ID NO:12, while the first heavy chain also includes a mutant Fc (hIgG1-Fc containing Q386C substitution), the CH3 sequence of which is set forth in SEQ ID NO:13.

In some embodiments of the fusion protein, the anti-PD1 antibody portion and the IL21 mutein are conjugated via a linker. In some embodiments, the linker comprises (GₙS)ₓ, wherein n is an integer between 2 and 4 inclusive, x is an integer between 1 and 4 inclusive. In some embodiments, the anti-PD1 antibody portion is directly connected to the IL21 mutein without a linker.

In some embodiments of the fusion protein, the first heavy chain comprises a sequence selected from the group consisting of SEQ ID NOS:17-25, e.g., SEQ ID NOS: 20, 22, 23, 24, and 25. The IL21 mutein conjugated with the first heavy chain can comprise a sequence selected from the group consisting of SEQ ID NOS:2-6, e.g., SEQ ID NO:6.

In some embodiments, the first heavy chain comprises a Fc region (CH3) comprising the sequence selected from the group consisting of SEQ ID NOS:17-22. In some of these embodiments, the first heavy chain can comprise a VH having the sequence of SEQ ID NO:7 or 11, and the second heavy chain can comprise a VH having the sequence of one of SEQ ID NOS:8-10, e.g., SEQ ID NO:10. The IL21 mutein conjugated with the first heavy chain can comprise a sequence selected from the group consisting of SEQ ID NOS:2-6, e.g., SEQ ID NO:6.

In a further aspect, the present disclosure provides a nucleic acid molecule encoding the any of the fusion protein described herein. A host cell (e.g., a CHO cell, a human embryonic kidney cell, a lymphocytic cell, or microorganisms, such as E. coli, and fungi, such as yeast) containing an expression vector containing the nucleic acid molecule, can be used to produce the fusion proteins of the present disclosure, preferably fusion proteins with monoclonal composition.

In one embodiment, DNA encoding partial or full-length fusion protein of the present disclosure (e.g., including the HC1, HC2, LC, and the IL-21 mutein attached to HC1) can be obtained by standard molecular biology techniques is inserted into one or more expression vectors such that the genes are operatively linked to transcriptional and translational regulatory sequences. The term “operatively linked” is intended to mean that a fusion protein encoding DNA sequence is ligated into a vector such that transcriptional and translational control sequences within the vector serve their intended function of regulating the transcription and translation of the fusion protein encoding DNA sequence. The term “regulatory sequence” is intended to include promoters, enhancers and other expression control elements (e.g., polyadenylation signals) that control the transcription or translation of the fusion protein encoding DNA sequences. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or B-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The fusion protein encoding DNA can be inserted into the expression vector. The recombinant expression vector can encode a signal peptide that facilitates secretion of the fusion protein from a host cell. The fusion protein encoding DNA can be cloned into the vector such that the signal peptide is linked in-frame to the amino terminus of the fusion protein encoding DNA. The signal peptide can be an immunoglobulin signal peptide or a heterologous signal peptide (i.e., a signal peptide from a non-immunoglobulin protein).

In further aspect, the present disclosure provides a pharmaceutical composition comprising one or more of the fusion proteins of the invention and a pharmaceutically acceptable carrier. As used herein, “pharmaceutically acceptable carrier” includes pharmaceutically acceptable carriers, excipients or stabilizers. These include but are not limited solvents, dispersion media, coatings, isotonic and absorption delaying agents, surface active agents, thickening or emulsifying agents, solid binders, dispersion or suspension aids, solubilizers, coatings, disintegrating agents, lubricants, isotonic agents, and the like that are physiologically compatible. The selection of suitable carrier is within the knowledge of an artisan skilled in the art. The composition may comprise one or more additional pharmaceutically active ingredients, such as another antibody, fusion protein, or a drug, e.g., a cytotoxic or anti-tumor agent. The pharmaceutical compositions of the invention also can be administered in a combination therapy with, for example, another anti-cancer agent, another anti-inflammatory agent, etc.

The pharmaceutical composition can be suitable for intravenous, intramuscular, subcutaneous, parenteral, epidermal, and other routes of administration. Depending on the route of administration, the active ingredient can be coated with a material or otherwise loaded in a material or structure to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the composition of the invention can be administered via a non-parenteral route, such as a topical, epidermal or mucosal route of administration, e.g., intranasally, orally, vaginally, rectally, sublingually or topically.

Methods of treatment are additionally provided by the present disclosure. The method, in exemplary embodiments, is a method of treating a subject in need thereof, comprising administering to the subject in need thereof a fusion protein or a pharmaceutical composition of the present disclosure in an amount effective to treat the subject. In exemplary aspects, the subject has a tumor (e.g., a solid tumor, a hematological malignancy, or a lymphoid malignancy) and the pharmaceutical composition is administered to the subject in an amount effective to treat the tumor in the subject. The tumor can include lung cancers, head and neck cancers, kidney cancers, breast cancers, brain cancers, melanoma and other skin cancers, ovarian cancers, liver cancers, pancreatic cancers, colon cancers, prostate cancers, gastrointestinal cancers, bladder cancers, blood cancers, lymphomas testicular cancers, gynecological cancers, and sarcomas. In some embodiments, the subject has a chronic viral infection, which can be HBV infection, HIV infection, or infection caused by Herpes Simplex Virus Type 1 (HSV-1), Herpes Simplex Virus Type 1 (HSV-2), Kaposi's Sarcoma-Associated Herpesvirus (KSHV), Human Herpesvirus 7 (HHV-7), Human Herpesvirus 6 (HHV-6), Cytomegalovirus (CMV), Epstein-Barr Virus (EBV), Varicella-Zoster Virus (VZV), Hepatitis D virus (HDV) and/or human papilloma virus (HPV)

### Example 1

Unless otherwise noted, all VH of anti-PD1 (wt and mutant) potion of the fusion proteins in this example is Keytruda VH or mutants based on mutations of Keytruda VH, and the IL-21 or IL21 mutein is directly attached to the C terminus of only the first heavy chain of the anti-PD-1 antibody, as shown in the FIGS. 1-2.

### Methods

### Human and Cyno PD-1 Binding Affinity

PD-1 binding affinity was determined by Biolayer Interferometry (BLI). Binding experiments were performed on Gator Bio Prime at 30° C. (Gatorbio.com/products/instruments). Anti-PD-1/IL-21 or anti-PD-1/IL-21 muteins were loaded onto anti-human IgGFc (HFC) biosensor tips. The biosensor tips were incubated with a serial dilution of either human or cynomolgus macaque PD-1 antigen. Kinetic constants were calculated using a monovalent (1:1) model.

### Human and Cyno IL21R Binding Affinity

IL-21R binding affinity was determined by BLI. Binding experiments were performed on Gator Bio Prime at 30° C. (Gatorbio.com/products/instruments). Human or cynomolgus macaque IL-21R protein loaded onto anti-His biosensor tips. The biosensor tips were incubated with fusion protein (anti-PD-1 antibody/IL21 (or IL21 mutein)) samples in a 3-fold serial dilution (six, 3-fold dilutions starting from 300 nM). Kinetic constants were calculated using a monovalent (1:1) model.

### PD-1 Reporter Assay

PD-1/PD-L1 checkpoint activity was measured using a PD-1/PD-L1 Blockade bioassay (Promega, J1250). To evaluate checkpoint activity, GloResponse Jurkat NFAT-luc2/PD-1 stable effector cells (Promega, #CS187102) and the CHO PD-L1 stable cell line (Promega, #CS178103) were co-cultured at a ratio of 1.25:1. Serially diluted antibodies/fusion proteins were added in triplicate and incubated for 6 hours at 37° C. and 5% CO₂. Luminescence was measured using the Bio-Glo Luciferase Assay System (Promega, #G7940). The ability to block PD-1/PD-L1 interaction is measured as an increase in Jurkat NFAT activation and can be quantified as EC₅₀ from a dose titration curve.

### HEK-Blue IL-21R Signaling Assay

HEK-Blue IL-21 reporter cells have been specifically engineered to overexpress IL-21R and activate JAK/STAT signaling (phosphorylation of STAT3) leading to transcriptional activation of the secreted embryonic alkaline phosphatase (SEAP) reporter gene. This activation results in the secretion of SEAP, which can be readily monitored using Quanti-Blue solution (a SEAP detection medium). To test functional IL-21R signaling, free WT human IL-21, anti PD-1/IL-21 or anti-PD-1/IL-21muteins were co-cultured with HEK-Blue IL-21 reporter cells and potency of response was determined by EC₅₀ from a dose titration curve. To test the selectivity of PD-1 signaling, HEK-Blue IL-21R cells were transiently co-transfected with human PD-1 and anti PD-1/IL-21muteins were co-incubated with PD-1 overexpressing HEK-Blue IL-21R cells and EC₅₀ was calculated from a dose titration curve.

Hut78 T cell line IL-21R/pSTAT3 Signaling Assay

HuT78 (ATCC, TIB-161) are a human T-cell line expressing the human IL-21R. To evaluate the potency and selectivity of anti-PD-1/IL-21mutein antibody cytokine fusions, Hut78 parental cells were stably transfected with human PD-1 to generate a Hut78-PD-1 cell line. Hut78 parental and Hut78PD-1 cells were treated with controls and antibody cytokine fusion in a dose titration and evaluated for phosphor-STAT3 Tyr705 levels as measured using the AlphaLISA Surefire Ultra pSTAT3 (Tyr705) Assay Kit (Perkin Elmer, #ALSU-PST3-A10K). EC₅₀ was calculated from a dose titration curve.

### In Vivo MC38 Colorectal Mouse Tumor Efficacy Model

The ability of a prototypic PD-1/IL-21 mutein antibody fusion to function in vivo was tested in a syngeneic MC38 mouse colorectal cancer tumor model. Mice engineered to with the extracellular domain of human PD-1 and IL-21R genetically coupled with the transmembrane and signaling domains of mouse PD-1 and IL-21R were used as a host to confer human binding specificity and biologically relevant cellular expression and signaling (B-hPD-1 plus/hIL21R (C57BL/6-Pdcd1^{tm3/(PDCD1)}Il21r^{tm1(IL21R)}/Bcgen); Biocytogen, Boston, MA U.S.A.).

B-hPD-1 plus/hIL-21R mice were subcutaneously inoculated with the mouse colorectal tumor, expressing human PD-1, hPD-1MC38, and tumors were allowed to grow to ˜ 100 mm3 prior to inoculation with the PD-1/IL-21mutein antibody fusion or a relevant isotype control antibody. Antibodies were dosed at day 0, 3, and 6 post 100 mm3 growth with 2.5 mg/kg at each administration. Animals were monitored for body weight loss, signs of morbidity and mortality, and tumor growth inhibition (TGI (%)=[1−(Tt−T0)/(Ct−C0)]×100%). The study was terminated on the same day when either the mean TV of the control group reaches approximately 2000 mm3, or on the last observation day, whichever comes first

### Results

### PD-1 Binding

The binding affinities of anti-PD-1/IL-21 and anti-PD-1/IL-21 mutein fusion antibodies to human PD-1 were measured by BLI (Table 1). The anti-PD-1 monoclonal antibody parental control, Keytruda, bound to human PD-1 with single digit nM affinity (Table 1; 4 nM). As expected, mutant fusion proteins which retain the parental anti-PD-1 binding domain but contain either K72Y, 75-80 deletion, or K72Y/75-80 deletion bound human PD-1 (Table 1; range 1.5-3 nM) with comparable affinity to the Keytruda control. Additional mutants with K72M/75-80 deletion or K72Q/75-80 deletion or with additions of the R+E, R+EE, or Cysteine mutations all retained comparable single digit nM binding to human PD-1 (Table 1; range 2.1-2.7 nM). Note that in this Example, R+E mutations denote the combination of Q87R mutation on the VH of the first chain and ELVS+E mutations on the second chain, as shown in FIG. 2d; R+EE mutations denote the combination of Q87R mutation on the VH of the first chain and ELVS+EE mutations on the second chain, as shown in FIG. 2e; and Cysteine mutations refer to the combination of mutations shown in FIG. 2f.

### IL-21R Binding

The binding affinities of anti-PD-1/IL-21 and anti-PD-1/IL-21mutein fusion antibodies to human IL-21R were measured by BLI (Table 2). Wild-type (wt) IL-21 bound tightly to IL-21R with sub-nM affinity (Table 2, 0.1 nM). This was comparable to the PD-1/IL-21 antibody cytokine fusion retaining the wt IL-21 sequence which was within 5-fold of the affinity of wt IL-21 (Table 1, 0.5 nM). IL-21 K72Y, 75-80 deletion, and K72Y/75-80 deletion mutein mutations were engineered to reduce affinity to the IL-21R. As expected, the anti-PD-1/IL-21 mutein fusion antibodies containing these mutations, showed a range of reduced affinity to IL-21R (Table 2; 6-152 nM) or a range of 60-1520-fold reduction in affinity versus wt-IL-21. In addition, further mutations, K72M and K72Q in combination with 75-80 deletion demonstrated optimal affinity range (Table 2; 12-34 nM (120-340-fold reduction vs wt IL-21). Additional mutations of R+E, R+EE, or Cysteine, did not alter the affinity range of these molecules (Table 2).

### PD-1 Checkpoint Blockade Reporter Assay

Antibodies that retained good PD-1 binding and optimal IL-21R binding profiles were evaluated for the ability to block the PD-1/PD-LI checkpoint in a Jurkat-NFAT reporter assay. The ability to break the PD-1/PD-L1 checkpoint is demonstrated by increased NFAT signaling and luciferase detection in the assay. Table 3 shows the half maximal effective concentration (EC₅₀) values of antibody and antibody cytokine fusion molecules tested in the assay. As expected, fusion molecules containing K72Y or K72Q alone, or paired with 75-80 del retained the ability to block PD-1/PD-L1 with EC₅₀ values within two-fold of the Keytruda parental control (Table 3; range 1.1-2 nM (ETYY); 2.4-4.7 nM (KIH)). The K72Y/75-80 deletion on the KIH background displayed the largest shift (Table 3; 11 nM). Pairing of the IL-21mutein mutations with pI reducing, R+E, R+EE, or heterodimeric pairing, Cysteine mutants, did not alter the ability to inhibit PD-1/PD-L1 checkpoint blockade (Table 3; 1.1-<2*). Thus anti PD-1/IL-21 mutein fusion molecules retain the original checkpoint blockade characteristics of the parental Keytruda antibody. Marked asterisk represents expected data.

HEK293 pSTAT3 IL-21R Activation Assay

To test the ability of anti PD-1/IL-21 antibody cytokine fusions to selectively activate IL-21R signaling in the presence of PD-1, HEK293 cells engineered to express the human IL-21R alone (HEK parental) or in combination with human PD-1 (HEK-PD-1) were tested for response to IL-21 signaling in a phosphor-STAT3 reporter assay. Free WT-IL-21 is utilized as a positive control in these assays and is expected to activate PD-1 negative and PD-1 positive cell lines with similar EC₅₀ potency. As shown in tables 4 and 5, WT-IL-21 activated pSTAT3 signaling in HEK Parental (Table 4; EC₅₀ of 0.06 nM) and HEK-PD-1 (Table 5; EC₅₀ of 0.05 nM) with similar potency. In comparison, the PD-1/IL-21 molecule comprised of WT-IL-21 showed a similar potency profile on HEK parental (Table 4; EC₅₀ of 0.01 nM) and HEK-PD-1 (Table 5; EC₅₀ of 0.03 nM). PD-1/IL-21muteins comprised of the K72Y, 75-80 deletion, or K72Y/75-80 deletion and showed varying reductions in EC₅₀ values compared to WT-IL-21 or PD-1/IL-21wt (Table 4; range EC₅₀ of 0.2 nM->100 nM) as engineered and in line with their affinity to the IL-21R (Table 2).

Hut78 pSTAT3 IL-21R Activation Assay

To better understand the ability of PD-1/IL-21 muteins to activate cells with endogenous IL-21R expression, phosphorylated STAT3 was monitored in the human T-cell line Hut78 expressing low-level IL-21R alone (Hut78 Parental) or low-level IL-21R in the presence of overexpressed human PD-1 (Hut78-PD-1). As shown in tables 4 and 5, WT-IL-21 activated pSTAT3 signaling in Hut78 Parental cells (Table 6; EC₅₀ of 0.09 nM) and Hut78-PD-1 (Table 7; EC₅₀ of 0.11 nM) with similar potency. A PD-1/WTIL-21 antibody fusion containing the wild-type IL-21 sequence showed similar potency to the free WT-IL-21 cytokine in Hut78 Parental cells (Table 6; EC₅₀ of 0.7 nM) and Hut78-PD-1 (Table 7; EC₅₀ of 0.12 nM). In comparison, PD-1/IL-21 muteins comprised of the K72Y/75-80, K72M/75-80, or K72Q/75-80 deletion in the presence or absence of pI reduction mutations, R+E or R+EE, or the heterodimeric pairing Cysteine mutations, did not display any potency on Hut78 parental (Table 6; EC₅₀>100 nM). In contrast, all PD-1/IL-21 muteins tested showed similar potency within 10-fold compared to WT-IL-21 on Hut78-PD-1 cells (Table 7; EC₅₀ range 0.1-0.9). These data support the ability of PD-1/IL-21Rmuteins to selectively activate IL-21R signaling (no effect, >100 nM EC₅₀) on endogenous IL-21R/PD-1 negative cells (Hut78) version comparable signaling to WT-IL-21 on IL-21R positive/PD-1 positive, Hut78-PD1 (0.1-0.9 nM EC50) >and showed varying reductions in EC₅₀ values compared to WT-IL-21 or PD-1/IL-21wt (Table 4; range EC₅₀ of 0.2 nM->100 nM) as engineered and in line with their affinity to the IL-21R (Table 2).

### In Vivo MC38 Colorectal Mouse Tumor Efficacy Model

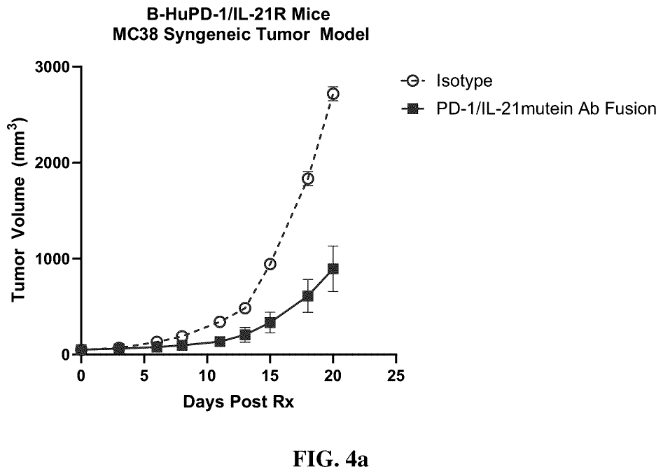

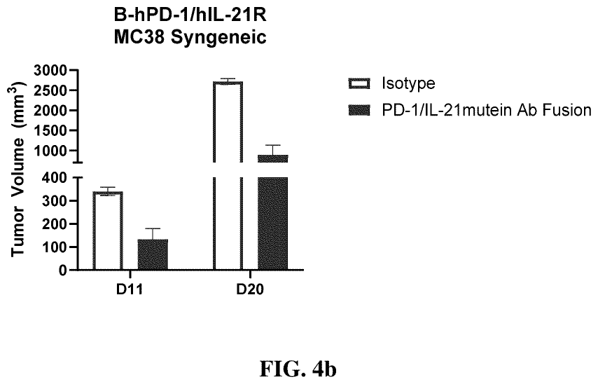

MC38 tumors grew at expected rates in B-hPD-1 plus/hIL21R mice and showed steady growth in the presence of the IgG1 isotype control antibody, reaching tumor volume (TV) (mean±SEM) of 2090.25±629.62 mm3 on Day 20 (FIG. 4a, 4b; TABLE 8). All Isotype treated animals had to be euthanized per the protocol based on morbidity and tumor burden by day 20. In contrast, treatment with the PD-1/IL-21 mutein antibody fusion, resulted in significant tumor control with TV (mean±SEM) of 893.48±237.81 mm3 on Day 20 (FIG. 4a, 4b; TABLE 8). This corresponded to a 58.66% tumor control compared to the Isotype (FIG. 4a, Xb; TABLE 8). All animals treated with the PD-1/IL-21 mutein antibody fusion responded to treatment with 1 complete response TV=0 mm3 observed (TABLE 8).

### Data Tables

### PD-1 Binding

TABLE 1
IL-21, IL21 mutein, and fusion proteins (anti-
PD-1/IL-21 muteins) binding to human PD-1.

| | PD-1 |
|---|---|
| | Binding |
| Proteins | (nM) |
| Keytruda | 4 |
| anti PD1-wt IL-21 | 3 |
| ETYY_anti PD1-IL-21 K72Y (FIG. 1d) | 3 |
| ETYY_anti PD1-IL-21 ΔK75-80 (FIG. 1e) | 3.7 |
| ETYY_anti PD1-IL-21 K72Y&ΔK75-80 (FIG. 1f) | 2.7 |
| KIH_anti PD1-IL-21 K72Y (FIG. 1a) | 1.5 |
| KIH_anti PD1-IL-21 ΔK75-80 (FIG. 1b) | 2.5 |
| KIH_anti PD1-IL-21 K72Y&ΔK75-80 (FIG. 1c) | 2.5 |
| ETYY_anti PD1-IL-21 K72M&ΔK75-80 (FIG. 2b) | 2.1 |
| ETYY_anti PD1-IL-21 K72Q&ΔK75-80 (FIG. 2c) | 2.7 |
| ETYY_anti PD1-IL-21 R + E | 2.5 |
| K72Q&ΔK75-80 (FIG. 2d) | |
| ETYY_anti PD1-IL-21 R + EE | 2.7 |
| K72Q&ΔK75-80 (FIG. 2e) | |
| ETYY_anti PD1-IL-21 R + EE | 2.3 |
| Cys K72Q&ΔK75-80 (FIG. 2f) | |

### IL-21R Binding

TABLE 2
IL-21, IL21 mutein, and fusion proteins (anti-
PD-1/IL-21 muteins) binding to human IL-21R.

| | IL-21R | |
|---|---|---|
| | Binding | Fold vs |
| Proteins | (nM) | wt IL-21 |
| wt IL-21 | 0.1 | N/A |
| anti PD1-wt IL-21 | 0.5 | 5 |
| ETYY_anti PD1-IL-21 K72Y (FIG. 1d) | 6 | 60 |
| ETYY_anti PD1-IL-21 ΔK75-80 (FIG. 1e) | 29 | 290 |
| ETYY_anti PD1-IL-21 K72Y&ΔK75-80 (FIG. 1f) | 111 | 1111 |
| KIH_anti PD1-IL-21 K72Y (FIG. 1a) | 92 | 920 |
| KIH_anti PD1-IL-21 ΔK75-80 (FIG. 1b) | 152 | 1520 |
| KIH_anti PD1-IL-21 K72Y&ΔK75-80 (FIG. 1c) | 12 | 120 |
| ETYY_anti PD1-IL-21 K72M&ΔK75-80 (FIG. 2b) | >19 | 190 |
| ETYY_anti PD1-IL-21 K72Q&ΔK75-80 (FIG. 2c) | 18 | 180 |
| ETYY_anti PD1-IL-21 R + E | 34 | 340 |
| K72Q&ΔK75-80 (FIG. 2d) | | |
| ETYY_anti PD1-IL-21 R + EE | 6 | 60 |
| K72Q&ΔK75-80 (FIG. 2e) | | |
| ETYY_anti PD1-IL-21 R + EE | 15 | 150 |
| Cys K72Q&ΔK75-80 (FIG. 2f) | | |

### PD-1 Reporter Activity

TABLE 3
IL-21, IL21 mutein, and fusion proteins (anti-
PD-1/IL-21 muteins) binding to human PD-1.

| | PD1/PD-L1 |
|---|---|
| Proteins | Block EC50 (nM) |
| Keytruda | 0.9 |
| anti PD1-wt IL-21 | 0.4 |
| ETYY_anti PD1-IL-21 K72Y (FIG. 1d) | 2 |
| ETYY_anti PD1-IL-21 ΔK75-80 (FIG. 1e) | 1.1 |
| ETYY_anti PD1-IL-21 K72Y&ΔK75-80 (FIG. 1f) | 1.9 |
| KIH_anti PD1-IL-21 K72Y (FIG. 1a) | 4.7 |
| KIH_anti PD1-IL-21 ΔK75-80 (FIG. 1b) | 11 |
| KIH_anti PD1-IL-21 K72Y&ΔK75-80 (FIG. 1c) | 2.4 |
| ETYY_anti PD1-IL-21 K72Q&ΔK75-80 (FIG. 2c) | 1.6 |
| ETYY_anti PD1-IL-21 R + E | 1.2 |
| K72Q&ΔK75-80 (FIG. 2d) | |
| ETYY_anti PD1-IL-21 R + EE | 1.1 |
| K72Q&ΔK75-80 (FIG. 2e) | |
| ETYY_anti PD1-IL-21 R + EE | <2* |
| Cys K72Q&ΔK75-80 (FIG. 2f) | |

pSTAT3 Activation—HEK293 Reporter

TABLE 4
In vitro activity of fusion proteins (anti-PD-1/IL-21
muteins) in HEK293 PD-1(negative) cell line

| | Stat3 | |
|---|---|---|
| | Activation | Fold vs |
| Proteins | (nM) | wt IL-21 |
| wt IL-21 | 0.06 | N/A |
| anti PD1-wt IL-21 | 0.01 | <1 |
| ETYY_anti PD1-IL-21 K72Y (FIG. 1d) | 0.2 | 3.3 |
| ETYY_anti PD1-IL-21 ΔK75-80 (FIG. 1e) | 1.4 | 23 |
| ETYY_anti PD1-IL-21 K72Y&ΔK75-80 | >100 | >1000 |
| (FIG. 1f) | | |
| KIH_anti PD1-IL-21 K72Y (FIG. 1a) | 1.1 | 18 |
| KIH_anti PD1-IL-21 ΔK75-80 (FIG. 1b) | 0.6 | 10 |
| KIH_anti PD1-IL-21 K72Y&ΔK75-80 (FIG. 1c) | >100 | >1000 |

TABLE 5
In vitro activity of fusion proteins (anti-PD-1/IL-21
muteins) in HEK293 PD-1 (positive) cell line

| | Stat3 | |
|---|---|---|
| | Activation | Fold vs |
| Proteins | (nM) | wt IL-21 |
| wt IL-21 | 0.05 | N/A |
| anti PD1-wt IL-21 | 0.03 | 1 |
| ETYY_anti PD1-IL-21 K72Y (FIG. 1d) | 0.1 | 5 |
| ETYY_anti PD1-IL-21 ΔK75-80 (FIG. 1e) | 0.4 | 20 |
| ETYY_anti PD1-IL-21 K72Y&ΔK75-80 | 0.4 | 20 |
| (FIG. 1f) | | |
| KIH_anti PD1-IL-21 K72Y (FIG. 1a) | 1.5 | 75 |
| KIH_anti PD1-IL-21 ΔK75-80 (FIG. 1b) | 0.2 | 10 |
| KIH_anti PD1-IL-21 K72Y&ΔK75-80 (FIG. 1c) | >100 | >1000 |

pSTAT3 Activation—Hut78 Human T-Cell Line

TABLE 6
In vitro activity of fusion proteins (anti-PD-1/IL-21
muteins) in Hut78 PD-1(negative) cell line

| | Stat3 | |
|---|---|---|
| | Activation | Fold vs |
| Proteins | (nM) | wt IL-21 |
| wt IL-21 | 0.09 | N/A |
| anti PD1-wt IL-21 | 0.07 | 2 |
| ETYY_anti PD1-IL-21 K72Y&ΔK75-80 | >100 | >1000 |
| (FIG. 2a) | | |
| ETYY_anti PD1-IL-21 K72M&ΔK75-80 | >100 | >1000 |
| (FIG. 2b) | | |
| ETYY_anti PD1-IL-21 K72Q&ΔK75-80 | >100 | >1000 |
| (FIG. 2c) | | |
| ETYY_anti PD1-IL-21 R + E | >100 | >1000 |
| K72Q&ΔK75-80 (FIG. 2d) | | |
| ETYY_anti PD1-IL-21 R + EE | >100 | >1000 |
| K72Q&ΔK75-80 (FIG. 2e) | | |
| ETYY_anti PD1-IL-21 R + EE | >100 | >1000 |
| Cys K72Q&ΔK75-80 (FIG. 2f) | | |

TABLE 7
In vitro activity of fusion proteins (anti-PD-1/IL-21
muteins) in Hut78 PD-1(positive) cell line

| | Stat3 | |
|---|---|---|
| | Activation | Fold vs |
| Proteins | (nM) | wt IL-21 |
| wt IL-21 | 0.11 | N/A |
| anti PD1-wt IL-21 | 0.12 | 1 |
| ETYY_anti PD1-IL-21 K72Y&ΔK75-80 | 0.23 | 2 |
| (FIG. 2a) | | |
| ETYY_anti PD1-IL-21 K72M&ΔK75-80 | 0.15 | 1 |
| (FIG. 2b) | | |
| ETYY_anti PD1-IL-21 K72Q&ΔK75-80 | 0.9 | 9 |
| (FIG. 2c) | | |
| ETYY_anti PD1-IL-21 R + E | 0.1 | 1 |
| K72Q&ΔK75-80 (FIG. 2d) | | |
| ETYY_anti PD1-IL-21 R + EE | 0.4 | 4 |
| K72Q&ΔK75-80 (FIG. 2e) | | |
| ETYY_anti PD1-IL-21 R + EE | 0.9 | 9 |
| Cys K72Q&ΔK75-80 (FIG. 2f) | | |

### B-hPD-1 Plus/hIL21R/MC38 In Vivo Tumor Model

TABLE 8
Tumor control by anti-PD-1/IL-21 mutein in a
humanized mouse model of colorectal cancer.

| | TV (mm3) | % | Complete |
|---|---|---|---|
| Proteins | Day 20 | TGI | Response |
| Isotype IgG1 | 2090.25 +/− 629.62 | — | — |
| Anti-PD-1/IL-21R mutein | 893.48 +/− 237.81 | 58.66 | 1 |
| antibody fusion | | | |
| (ETYY_anti PD1-IL-21 R + EE | | | |
| K72Q&ΔK75-80) (FIG. 2e) | | | |

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

SEQUENCE LISTING

SEQ ID NO: 1
Human wild-type IL-21:
QGQDRHMIRMRQLIDIVDQLKNYVNDLVPEFLPAPEDVETNCEWSAFSCFQKAQLKSANTGNNE
RIINVSIKKLKRKPPSTNAGRRQKHRLTCPSCDSYEKKPPKEFLERFKSLLQKMIHQHLSSRTHGSE
DS
SEQ ID NO: 2
Human mutant 1-IL-21: K72Y
QGQDRHMIRMRQLIDIVDQLKNYVNDLVPEFLPAPEDVETNCEWSAFSCFQKAQLKSANTGNNE
RIINVSIYKLKRKPPSTNAGRRQKHRLTCPSCDSYEKKPPKEFLERFKSLLQKMIHQHLSSRTHGSEDS
SEQ ID NO: 3
Human mutant 2-IL-21: 75-80 deletion
QGQDRHMIRMRQLIDIVDQLKNYVNDLVPEFLPAPEDVETNCEWSAFSCFQKAQLKSANTGNNE
RIINVSIKKLTNAGRRQKHRLTCPSCDSYEKKPPKEFLERFKSLLQKMIHQHLSSRTHGSEDS
SEQ ID NO: 4
Human mutant 3-IL-21: K72Y + 75-80 deletion
QGQDRHMIRMRQLIDIVDQLKNYVNDLVPEFLPAPEDVETNCEWSAFSCFQKAQLKSANTGNNE
RIINVSIYKLTNAGRRQKHRLTCPSCDSYEKKPPKEFLERFKSLLQKMIHQHLSSRTHGSEDS
SEQ ID NO: 5
Human mutant 3-IL-21: K72M + 75-80 deletion
QGQDRHMIRMRQLIDIVDQLKNYVNDLVPEFLPAPEDVETNCEWSAFSCFQKAQLKSANTGNNE
RIINVSIMKLTNAGRRQKHRLTCPSCDSYEKKPPKEFLERFKSLLQKMIHQHLSSRTHGSEDS
SEQ ID NO: 6
Human mutant 3-IL-21: K72Q + 75-80 deletion
QGQDRHMIRMRQLIDIVDQLKNYVNDLVPEFLPAPEDVETNCEWSAFSCFQKAQLKSANTGNNE
RIINVSIQKLTNAGRRQKHRLTCPSCDSYEKKPPKEFLERFKSLLQKMIHQHLSSRTHGSEDS
SEQ ID NO: 7
Keytruda VH (wt parental)
QVQLVQSGVEVKKPGASVKVSCKASGYTFTNYYMYWVRQAPGQGLEWMGGINPSNGGTNFNE
KFKNRVTLTTDSSTTTAYMELKSLQFDDTAVYYCARRDYRFDMGFDYWGQGTTVTVSS
SEQ ID NO: 8
Keytruda VH (pI reducing mutant (ELVS))
EVQLVQSGVELVKPGASVKVSCKASGYTFTNYYMYWVRQAPGQGLEWMGGINPSNGGTNFNE
KFKNRVTLTTDSSTTTAYMELSSLQFDDTAVYYCARRDYRFDMGFDYWGQGTTVTVSS
SEQ ID NO: 9
Keytruda VH (pI reducing mutant-2 (ELVS + E))
EVQLVQSGVELVKPGASVKVSCKASGYTFTNYYMYWVRQAPGQGLEWMGGINPSNGGTNFNE
KFKNRVTLTTDSSTTTAYMELSSLEFDDTAVYYCARRDYRFDMGFDYWGQGTTVTVSS
SEQ ID NO: 10
Keytruda VH (pI reducing mutant-2 (ELVS + EE))
EVQLVQSGVELVEPGASVKVSCKASGYTFTNYYMYWVRQAPGQGLEWMGGINPSNGGTNFNE
KFKNRVTLTTDSSTTTAYMELSSLEFDDTAVYYCARRDYRFDMGFDYWGQGTTVTVSS
SEQ ID NO: 11
Keytruda VH (pI reducing mutant-2 (HC1-Q87R (R in REE))
QVQLVQSGVEVKKPGASVKVSCKASGYTFTNYYMYWVRQAPGQGLEWMGGINPSNGGTNFNE
KFKNRVTLTTDSSTTTAYMELKSLRFDDTAVYYCARRDYRFDMGFDYWGQGTTVTVSS
SEQ ID NO: 12
hIgG1-Fc (CH3, Cysteine HC2 S400C)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPEVYTLPPSREEMTKNQVSLTCLVKGFYPSD
IAVEWESNGQPENNYKTTPPVLDCDGSFFLTSKLTVDKSRWQQGNVFSCSVMHEALHNHYTQK
SLSLSPGK
SEQ ID NO: 13
hIgG1-Fc (CH3, Cysteine HC1 Q386C)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPEVKTLPPYREEMTKNQVSLYCLVKGFYPS
DIAVEWESNGQPENCYKTTPPVLDSDGSFFLYSKLTVDKSRWQQGNVFSCSVMHEALHNHYTQ
KSLSLSPGK
SEQ ID NO: 14
KIH HC2
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTKNQVSLTCLVKGFYPS
DIAVEWESNGQPENNYKTTPPVLDSDGSFFLTSKLTVDKSRWQQGNVFSCSVMHEALHNHYTQ
KSLSLSPGK
SEQ ID NO: 15
KIH HC1
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTKNQVSLYCLVKGFYPS
DIAVEWESNGQPENNYKTTPPVLDSDGSFFLYSKLTVDKSRWQQGNVFSCSVMHEALHNHYTQ
KSLSLSPGK
SEQ ID NO: 16
ETYY HC2
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPEVYTLPPSREEMTKNQVSLTCLVKGFYPSD
IAVEWESNGQPENNYKTTPPVLDSDGSFFLTSKLTVDKSRWQQGNVFSCSVMHEALHNHYTQK
SLSLSPGK
SEQ ID NO: 17
ETYY HC1
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPYREEMTKNQVSLYCLVKGFYPS
DIAVEWESNGQPENNYKTTPPVLDSDGSFFLYSKLTVDKSRWQQGNVFSCSVMHEALHNHYTQ
KSLSLSPGK
SEQ ID NO: 18
ETYY HC1 mutA (Delete c-terminal K)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPYREEMTKNQVSLYCLVKGFYPS
DIAVEWESNGQPENNYKTTPPVLDSDGSFFLYSKLTVDKSRWQQGNVFSCSVMHEALHNHYTQ
KSLSLSPG
SEQ ID NO: 19
ETYY HC1 mutB (c-terminal K-R)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPYREEMTKNQVSLYCLVKGFYPS
DIAVEWESNGQPENNYKTTPPVLDSDGSFFLYSKLTVDKSRWQQGNVFSCSVMHEALHNHYTQ
KSLSLSPGR
SEQ ID NO: 20
ETYY HC1 mutC (c-terminal K-A)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPYREEMTKNQVSLYCLVKGFYPS
DIAVEWESNGQPENNYKTTPPVLDSDGSFFLYSKLTVDKSRWQQGNVFSCSVMHEALHNHYTQ
KSLSLSPGA
SEQ ID NO: 21
ETYY HC1 mutD(c-terminal K-Q)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPYREEMTKNQVSLYCLVKGFYPS
DIAVEWESNGQPENNYKTTPPVLDSDGSFFLYSKLTVDKSRWQQGNVFSCSVMHEALHNHYTQ
KSLSLSPGQ
SEQ ID NO: 22
ETYY HC1 mutG(Delete c-terminal K −S)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPYREEMTKNQVSLYCLVKGFYPS
DIAVEWESNGQPENNYKTTPPVLDSDGSFFLYSKLTVDKSRWQQGNVFSCSVMHEALHNHYTQ
KSLSLSPGS
SEQ ID NO: 23
ETYY HC1 mutE (Delete c-terminal K-A +G4S)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPYREEMTKNQVSLYCLVKGFYPS
DIAVEWESNGQPENNYKTTPPVLDSDGSFFLYSKLTVDKSRWQQGNVFSCSVMHEALHNHYTQ
KSLSLSPGAGGGGS
SEQ ID NO: 24
ETYY HC1 mutF (Delete c-terminal K +G4S)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPYREEMTKNQVSLYCLVKGFYPS
DIAVEWESNGQPENNYKTTPPVLDSDGSFFLYSKLTVDKSRWQQGNVFSCSVMHEALHNHYTQ
KSLSLSPGGGGGS
SEQ ID NO: 25
ETYY HC1 mutH (Delete c-terminal K +SG4S)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGGPSVFLFPPKPK
DTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPYREEMTKNQVSLYCLVKGFYPS
DIAVEWESNGQPENNYKTTPPVLDSDGSFFLYSKLTVDKSRWQQGNVFSCSVMHEALHNHYTQ
KSLSLSPGSGGGGS

1. A fusion protein comprising an anti-PD1 antibody portion having a C-terminus, and an IL21 mutein conjugated with the anti-PD1 antibody portion with its C-terminus, wherein the IL21 mutein contains at least one of the two mutations relative to the wild-type human IL21: (1) one of K72Y substitution, K72M substitution, and K72Q substitution, and (2) 75-80 deletion.

2. The fusion protein of claim 1, wherein the IL21 mutein contains both of (1) one of K72Y substitution, K72M substitution, and K72Q substitution, and (2) 75-80 deletion.

3. The fusion protein of claim 1, wherein the anti-PD1 antibody has an IgG1 format and comprises a first heavy chain and second heavy chain, the first heavy chain comprising a T366Y mutation and the second heavy chain comprising a Y407T mutation.

4. The fusion protein of claim 1, wherein the anti-PD1 antibody has an IgG1 format and comprises a first heavy chain and second heavy chain, the first heavy chain containing K360, S354Y and T366Y mutations, and the second heavy chain containing G347E, Y349 and Y407T mutations.

5. The fusion protein of claim 1, wherein the anti-PD1 antibody comprises a first heavy chain comprising a VH comprising a sequence of SEQ ID NO:7.

6. The fusion protein of claim 5, wherein the anti-PD1 antibody further comprises a second heavy chain comprising a VH comprising a sequence selected from the group consisting of the sequences of SEQ ID NOS:8-10.

7. The fusion protein of claim 1, wherein the anti-PD1 antibody comprises a first heavy chain comprising a VH comprising a sequence of SEQ ID NO:11.

8. The fusion protein of claim 7, wherein the anti-PD1 antibody further comprises a second heavy chain comprising a VH comprising a sequence selected from the group consisting of the sequences of SEQ ID NOS:8-10.

9. The fusion protein of any of claims 3-8, wherein the first heavy chain includes the sequence of SEQ ID NO:13, and the second heavy chain includes the sequence of SEQ ID NO:12.

10. The fusion protein of claim 9, wherein only the first heavy chain is conjugated with the IL21 mutein and the second heavy chain is not conjugated with any IL21 mutein.

11. The fusion protein of claim 1, wherein the anti-PD1 antibody portion and the IL21 mutein are conjugated via a linker.

12. The fusion protein of claim 11, wherein the linker comprises (GₙS)ₓ, wherein n is an integer between 2 and 4 inclusive, x is an integer between 1 and 4 inclusive.

13. The fusion protein of claim 1, wherein the anti-PD1 antibody portion is directly connected to the IL21 mutein.

14. The fusion protein of claim 9, wherein the first heavy chain comprises the sequence selected from the groups consisting of SEQ ID NOS:17-22.

15. The fusion protein of claim 14, wherein the second heavy chain comprises the sequence set forth in SEQ ID NO:16.

16. A nucleic acid encoding the fusion protein of any of claims 1-15.

17. A vector comprising the nucleic acid of claim 16.

18. A pharmaceutical composition comprising a fusion protein of any of claims 1-15 and a pharmaceutically acceptable carrier.

19. A method of treating a cancer of a subject, comprising administering to the subject an effective amount of a fusion protein of any of claims 1-15 or of the pharmaceutical composition of claim 18.

20. The method of claim 19, wherein the cancer is a solid tumor, a hematological malignancy, or a lymphoid malignancy.

21. The method of claim 19, wherein the cancer comprises lung cancers, head and neck cancers, kidney cancers, breast cancers, brain cancers, melanoma and other skin cancers, ovarian cancers, liver cancers, pancreatic cancers, colon cancers, prostate cancers, gastrointestinal cancers, bladder cancers, blood cancers, lymphomas testicular cancers, gynecological cancers, and sarcomas

22. A method of treating a chronic viral infection of a subject, comprising administering to the subject an effective amount of a fusion protein of any of claims 1-15 or of the pharmaceutical composition of claim 18.

23. The method of claim 22, wherein the chronic viral infection is caused by one or more of HBV, HIV, Herpes Simplex Virus Type 1 (HSV-1), Herpes Simplex Virus Type 1 (HSV-2), Kaposi's Sarcoma-Associated Herpesvirus (KSHV), Human Herpesvirus 7 (HHV-7), Human Herpesvirus 6 (HHV-6), Cytomegalovirus (CMV), Epstein-Barr Virus (EBV), Varicella-Zoster Virus (VZV), Hepatitis D virus (HDV), and human papilloma virus (HPV).


SEQUENCE LISTING (25 sequences)

SEQ ID NO: 1 - protein, 133 aa, Homo sapiens [sequence omitted]

SEQ ID NO: 2 - protein, 133 aa, synthetic construct
QGQDRHMIRMRQLIDIVDQLKNYVNDLVPEFLPAPEDVETNCEWSAFSCFQKAQLKSANTGNNERIINVSIYKLKRKPPSTNAGRRQKHRLTCPSCDSYEKKPPKEFLERFKSLLQKMIHQHLSSRTHGSEDS

SEQ ID NO: 3 - protein, 127 aa, synthetic construct
QGQDRHMIRMRQLIDIVDQLKNYVNDLVPEFLPAPEDVETNCEWSAFSCFQKAQLKSANTGNNERIINVSIKKLTNAGRRQKHRLTCPSCDSYEKKPPKEFLERFKSLLQKMIHQHLSSRTHGSEDS

SEQ ID NO: 4 - protein, 127 aa, synthetic construct
QGQDRHMIRMRQLIDIVDQLKNYVNDLVPEFLPAPEDVETNCEWSAFSCFQKAQLKSANTGNNERIINVSIYKLTNAGRRQKHRLTCPSCDSYEKKPPKEFLERFKSLLQKMIHQHLSSRTHGSEDS

SEQ ID NO: 5 - protein, 127 aa, synthetic construct
QGQDRHMIRMRQLIDIVDQLKNYVNDLVPEFLPAPEDVETNCEWSAFSCFQKAQLKSANTGNNERIINVSIMKLTNAGRRQKHRLTCPSCDSYEKKPPKEFLERFKSLLQKMIHQHLSSRTHGSEDS

SEQ ID NO: 6 - protein, 127 aa, synthetic construct
QGQDRHMIRMRQLIDIVDQLKNYVNDLVPEFLPAPEDVETNCEWSAFSCFQKAQLKSANTGNNERIINVSIQKLTNAGRRQKHRLTCPSCDSYEKKPPKEFLERFKSLLQKMIHQHLSSRTHGSEDS

SEQ ID NO: 7 - protein, 120 aa, synthetic construct [sequence omitted]

SEQ ID NO: 8 - protein, 120 aa, synthetic construct
EVQLVQSGVELVKPGASVKVSCKASGYTFTNYYMYWVRQAPGQGLEWMGGINPSNGGTNFNEKFKNRVTLTTDSSTTTAYMELSSLQFDDTAVYYCARRDYRFDMGFDYWGQGTTVTVSS

SEQ ID NO: 9 - protein, 120 aa, synthetic construct
EVQLVQSGVELVKPGASVKVSCKASGYTFTNYYMYWVRQAPGQGLEWMGGINPSNGGTNFNEKFKNRVTLTTDSSTTTAYMELSSLEFDDTAVYYCARRDYRFDMGFDYWGQGTTVTVSS

SEQ ID NO: 10 - protein, 120 aa, synthetic construct
EVQLVQSGVELVEPGASVKVSCKASGYTFTNYYMYWVRQAPGQGLEWMGGINPSNGGTNFNEKFKNRVTLTTDSSTTTAYMELSSLEFDDTAVYYCARRDYRFDMGFDYWGQGTTVTVSS

SEQ ID NO: 11 - protein, 120 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLRFDDTAVYYCARRDYRFDMGFDYWGQGTTVTVSS

SEQ ID NO: 12 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDCDGSFFLTSKLTVDKSRWQQGNVFSCSVMHEALHNHYTQKSLSLSPGK

SEQ ID NO: 13 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVKTLPPYREEMTKNQVSLYCLVKGFYPSDIAVEWESNGQPENCYKTTPPVLDSDGSFFLYSKLTVDKSRWQQGNVFSCSVMHEALHNHYTQKSLSLSPGK

SEQ ID NO: 14 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 15 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 17 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGR

SEQ ID NO: 20 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGA

SEQ ID NO: 21 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGQ

SEQ ID NO: 22 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGS

SEQ ID NO: 23 - protein, 335 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGAGGGGS

SEQ ID NO: 24 - protein, 334 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGGGGGS

SEQ ID NO: 25 - protein, 335 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPYREEMTKNQVSLYCLVKGFYPSDIAVEWESNGQPENNYKTTPPVLDSDGSFFLYSKLTVDKSRWQQGNVFSCSVMHEALHNHYTQKSLSLSPGSGGGGS